(19) United States
(12) Patent Application Publication
(10) Pub. No.: US 2007/0203452 A1
(43) Pub. Date: Aug. 30, 2007

(54) Platform Catheter

(76) Inventor: Bharat A. Mehta, Bloomfield, MI (US)

Correspondence Address: Kenneth I Kohn;Kohn & Associates, Suite 410, 30500 Northwestern Highway, Farmington Hills, MI, 48334, US

(21) Appl. No.: 10/575,059

(22) PCT Filed: Oct. 7, 2004
(86) PCT No.: PCT/US04/32866

§ 371 Date: Jan. 16, 2007

Related U.S. Application Data
(60) Provisional application No. 60/509,367, filed on Oct. 7, 2003.

(51) Int. Cl. A61M 29/04

(52) U.S. Cl. 604/96.01; 604/93.01

(57) ABSTRACT

A catheter including an end portion having a platform extending substantially radially outwardly therefrom. A catheter for treating aneurysms, the catheter including a lumen having an insertion end and an opposite end and a radially outwardly expandable ring attached to the insertion end of the catheter. An expandable ring capable of being attached to a catheter. A method of treating an aneurysm by inserting the above catheter into an artery in need of treatment.

### BACKGROUND OF THE INVENTION

1. Field of the Invention

The present invention generally relates to medical devices. More specifically, the present invention relates to catheters for use in treating aneurysms.

2. Description of Related Art

Cerebral aneurysms are enlargements of the cerebral vasculature, which protrude like a balloon from the wall of a cerebral artery. The cerebral aneurysm has a neck that leads to the parental vessel and a body or “dome” that can vary in diameter from 1-30 mm.

The wall of the aneurysm is often weak and can rupture, leading to hemorrhage. Rupture of the aneurysm can kill a patient or leave the patient with permanent or transitory mental and physical deficits.

Aneurysms are often treated in order to prevent rupture that can lead to hemorrhage, or to prevent rebleeding of acutely ruptured aneurysms. A conventional method of treating aneurysms is to fill the aneurysm with coils. The coils are introduced into the aneurysm one at a time through a delivery catheter until the aneurysm is filled. The aneurysm eventually becomes a solid mass of coils and thrombi.

A problem with the conventional method of using coils to fill aneurysms is that the aneurysm becomes a relatively solid mass due to coils and thrombi contained therein. The mass of coils and thrombi exert pressure on adjacent areas of the brain, which may lead to other problems. Another problem with the conventional method is that the coils must be delivered one at a time into the aneurysm, which increases the procedure time and risk to the patient. For large aneurysms, up to twenty coils may be required to fill the aneurysm.

The traditional method of treating patients with ruptured and unruptured cerebral aneurysms is surgical clipping. Approximately 15,000 of the surgical procedures are performed in the U.S. each year. Surgical mortality from clipping a previously ruptured cerebral aneurysm varies from 5% to 20%, depending upon the site of the aneurysm and the neurological condition of the patient at the time of surgery. Surgical mortality for an unruptured aneurysm is from 2% to 10%.

As a result of the high surgical mortality rate, a number of endovascular techniques have been developed to treat cerebral aneurysms. In 1974, Serbinenko first reported the successful treatment of intracranial aneurysms with detachable balloons. Using an endovascular approach similar to an angiogram, the balloon is directed under fluoroscopic guidance to the aneurysm. The balloon can be placed inside of the aneurysm, leaving the parent artery intact. If the neck of the aneurysm were too large to entrap the balloon completely inside of the aneurysm, occlusion of the parent vein or artery would have to be performed. Large aneurysms typically required multiple balloons. The endovascular approach typically includes two major steps. The first step involves the introduction of the catheter to the aneurysm site using devices such as shown in the Engelson patents. The second step often involves filling the aneurysm in some fashion or another. For instance, a balloon may be introduced into the aneurysm from the distal portion of the catheter where it is inflated, detached, and left to occlude the aneurysm. In this way, the parent artery is preserved. Balloons are becoming less in favor because of the difficulty in introducing the balloon into the aneurysm sac, the possibility of an aneurysm rupture due to overinflation of the balloon within the aneurysm, and the risk associated with the traction produced when detaching the balloon.

Since 1974, a variety of detachable and nondetachable balloons made of a variety of materials, especially silicone and biocompatible polymeric latices, have been introduced. However, most aneurysms do not have the round or elliptical configuration of a balloon. Consequently, large aneurysms had to be filled with multiple balloons, leaving dead space for continued aneurysmal filling and subsequent rupture. The unfilled volumes allow for the development of clot in the aneurysmal remnant, enabling embolization to produce a stroke. Migration of a balloon from the aneurysm into the parent artery, or to a more distal branch of the parent system to, produce a stroke, has also been reported in the literature. The use of balloons for direct aneurysm occlusion is therefore no longer performed. Parent artery occlusion using a detachable balloon is still a viable procedure, although the blood flow to the hemisphere may be compromised with such a procedure, producing a stroke.

Aneurysmal occlusion with microcoils is another endovascular technique. Very soft platinum microcoils have been developed recently. The microcoils can be formed both with and without fibers that induce thrombus formation. The soft microcoils are placed directly into an aneurysm, and the degree of occlusion is related to the ability to pack the coil mass tightly. A highly desirable embolism-forming device which may be introduced into an aneurysm using endovascular placement procedures, is found in U.S. Pat. No. 4,994,069, to Ritchart et al. There is described a device—typically a platinum/tungsten alloy coil having a very small diameter—which may be introduced into an aneurysm through a catheter such as those described in Engelson above. These coils are often made of wire having a diameter of 2-6 mils. The coil diameter may be 10-30 mils. These soft, flexible coils may be of any length desirable and appropriate for the site to be occluded. For instance, the coils may be used to fill a berry aneurysm. Within a short period of time after the filling of the aneurysm with the embolic device, a thrombus forms in the aneurysm and is shortly thereafter complemented with a collagenous material which significantly lessens the potential for aneurysm rupture.

Coils such as seen in Ritchart et al. may be delivered to the vasculature site in a variety of ways including, e.g., mechanically detaching them from the delivery device as is shown in Palermo (U.S. Pat. No. 5,250,071) or by electrolytic detachment as is shown in Guglielmi et al. (U.S. Pat. No. 5,122,136) as was discussed above.

Guglielmi et al. shows an embolism-forming device and procedure for using that device. Specifically, Guglielmi et al. fills a vascular cavity such as an aneurysm with an embolic device such as a platinum coil which coil has been endovascularly delivered. The coil is then severed from its insertion tool by the application of a small electric current. Desirably, the insertion device involves a guidewire which is attached at its distal end to an embolic device by an electrolytic, sacrificial joint. Guglielmi et al. suggests that when the embolic device is a platinum coil, the platinum coil may be 1-50 cm. or longer as is necessary. Proximal of the embolic coil is a guidewire, often stainless steel in construction. The guidewire is used to push the platinum embolic coil, obviously with great gentleness, into the vascular site to be occluded. The patent shows a variety ways of linking the embolic coil to the pusher guidewire. For instance, the guidewire may be tapered at its distal end and the distal tip of the guidewire is soldered into the proximal end of the embolic coil. Additionally, a stainless steel coil is wrapped coaxially about the distal tapered portion of the guidewire to provide column strength to the guidewire. This coaxial stainless steel wire is joined both to the guidewire and to the embolic coil. Insulation may be used to cover a portion of the strength-providing stainless steel coil. This arrangement provides for two regions which must be electrolytically severed before the embolic coil is severed from the guidewire.

A further variation of the Guglielmi detachable coil is one in which the distal tip of the stainless steel guidewire is not soldered to the proximal end of the embolic device. A simple conical stainless steel wire is included from the stainless steel guidewire to the embolic coil.

A further variation found in Guglielmi et al. includes a thin, threadlike extension between the guidewire core and the proximal end of the embolic coil. In this way, the guidewire does not extend to the embolic coil, but instead relies upon a separately introduced extension.

A continuation-in-part application to Guglielmi et al. '136 discussed above. U.S. Pat. No. 5,354,295, issued Oct. 11, 1994, entitled “Improvements in an Endovascular Electrolytically Detachable Wire and Tip for the Formation of Thrombus in Arteries, Veins, Aneurysms, Vascular Malformations and Arteriovenous Fistulas” describes the use of mechanically detachable embolic devices as well as those that are electrolytically detachable. The embolic devices may be augmented with attached filaments.

Aneurysms having wide necks relative to their diameters may not be even treatable with this technique. A wide neck allows the coils to herniate into the parent artery, which may produce unwanted parent artery occlusion and stroke. Therefore, aneurysms with wide necks usually must be treated surgically, with a higher morbidity/mortality rate than if an endovascular method had been available. The GDC also produces undesirable artifacts on MR scans, making it impossible to define an aneurysmal remnant or tissue injury in the region of the aneurysm. Lastly, electrolytic detachment of the GDC can result in migration of the solder remnants into the intracranial circulation.

Another procedure, the extra-intravascular approach, involves surgically exposing or stereotaxicly reaching an aneurysm with a probe. The wall of the aneurysm is then perforated from the outside and various techniques are used to occlude the interior in order to prevent it from re-bleeding. The techniques used to occlude the aneurysm include electrothrombosis, adhesive embolization, hog hair embolization, and ferromagnetic thrombosis. These procedures are discussed in U.S. Pat. No. 5,122,136 to Guglielmi et al.

Balloons need to be inflated periodically to prevent acute ischemia in the distal territory of the parent artery. Currently, this is a well accepted method of endovascular treatment of wide neck aneurysm. However, this is by no means easy or safe all the time and requires significant expertise and experience eon the part of the operator.

It would therefore be useful to develop new methods and devices for treating aneurysms.

### SUMMARY OF THE INVENTION

According to the present invention, there is provided a catheter including an end portion having a platform extending substantially radially outwardly therefrom. A catheter for treating aneurysms, the catheter including a lumen having an insertion end and an opposite end and a radially outwardly expandable ring attached to the insertion end of the catheter is provided. Also provided is an expandable ring capable of being attached to a catheter. A method of treating an aneurysm by inserting the above catheter into an artery in need of treatment is also provided.

### DESCRIPTION OF THE DRAWINGS

Other advantages of the present invention are readily appreciated as the same becomes better understood by reference to the following detailed description, when considered in connection with the accompanying drawings wherein:

FIG. 1 shows the catheter inserted in situ; and

FIGS. 2A and B show the catheter of the present invention both in the unexpanded (FIG. 2A) and expanded (FIG. 2B) condition.

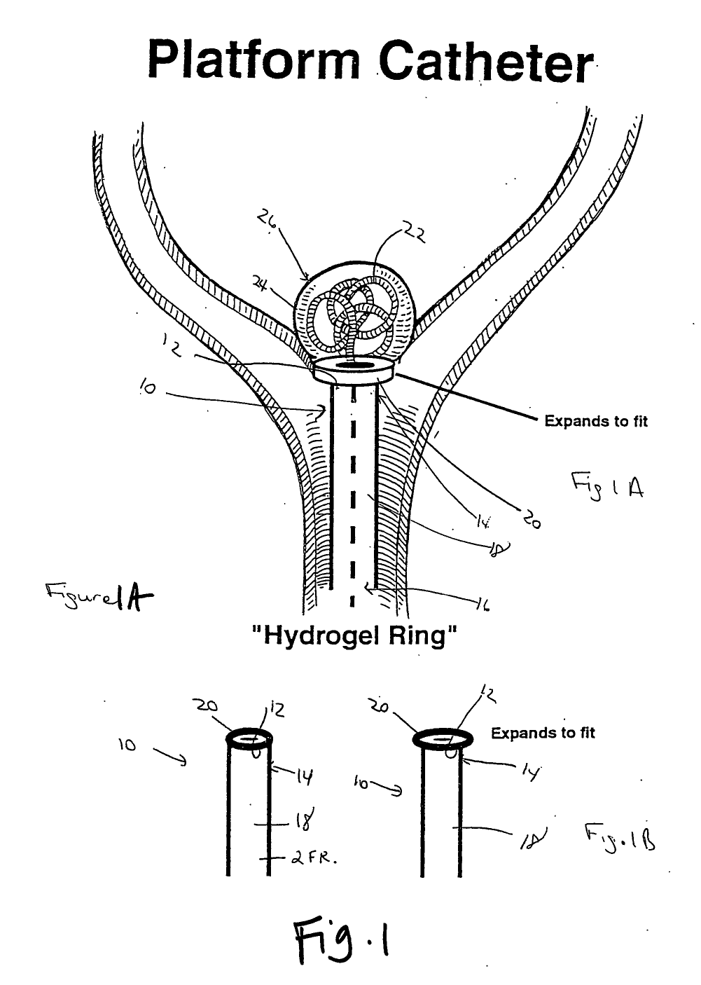

### DESCRIPTION OF THE INVENTION

Generally, the present invention provides a platform catheter, generally shown as 10, and method of using the same. The catheter 10 includes a modified microcatheter tip 12. The parts of the catheter 10 are made of materials known to those of skill in the art that are sufficient to perform the method of the present invention. The catheter 10 is preferably formed from a polymeric material or other similar materials known to those of skill in the art.

The catheter 10 of the present invention can be any catheter known to those of skill in the art. The catheter 10 includes an insertion end 14, an opposite end 16, and a lumen 18 extending therebetween. A modified tip 12 includes a ring 20, formed of a hydrophilic material, which is attached to the insertion end 14 of the catheter 10. The tip 12 extends radially outwardly from the catheter 10, forming a platform structure. The hydrophilic material is able to expand up to three times its original dimensions, thereby expanding the platform. The ring 20 creates a sufficient platform at the base of a wide neck aneurysm. The insertion end 14 of the catheter 10 is made of a material known to those of skill in the art to be sufficient to create a platform.

The catheter 10 of the present invention is very simple yet quite useful because it eliminates the use of a 2^{nd} balloon catheter. The ring 20 of hydrophilic material is attached to the tip 12 of the microcatheter 10, which is capable of delivering platinum coils 22 into the sac 24 of the wide neck aneurysm 26. The ring material swells to a larger diameter compared to the initial dehydrated state. The enlarged ring 20 creates a platform that provides sufficient support to the coil mass 22 in the sac 24 of the wide neck aneurysm 26 and blocks/prevents migration of coil loops 22 into the parent artery, thereby obviating the need for an additional balloon catheter.

An existing catheter 10 can be modified at the tip 12. To do so, a ring 20 of hydrophilic material is attached to the tip 10 of the microcatheter 12. The ring 20 can expand two to three times its original dimension in situ, thereby providing sufficient platform at the base of the wide neck aneurysm 26 while initially allowing for easy insertion.

The present invention provides a treatment of abnormalities in a patient's vascular system. A specific use of the catheter 10 of the present invention is for the treatment of cerebral aneurysms, although the various aspects of the invention described below also may be useful in treating other abnormalities such as arteriovenous malformations (AVM), hypervascular tumors, cavernous carotid fistulas, fibroid tumors, and non-reversible sterilization via fallopial occlusion.

Endovascular techniques treat aneurysms using a microcatheter positioned within the aneurysm or the parent artery. U.S. Pat. No. 5,122,136 to Guglielmi et al. describes one known endovascular technique. Other techniques known to those of skill in the art can be used to treat an aneurysm using the device of the present invention.

Additionally, the treatment can include inserting platinum coils 22 at the location of the aneurysm. The soft pliable coil 22 can be made from platinum or a platinum alloy that is soldered to a stainless steel coil and push wire. The stainless steel coil and push wire are used to position the platinum coil in the dome of the aneurysm, and position the junction between platinum coil and stainless steel coil near the neck of the aneurysm. A direct current (DC) is applied to the push wire, stainless steel coil and platinum coil to form a thrombogenic mass within the dome and thereby occlude the aneurysm.

By exposing the junction between the platinum coil and its push wire coil to blood and continuing to apply electric current to the push wire, the exposed portion of the stainless steel coil electrolytically dissolves. The remaining portion of the stainless steel coil and push wire then can be withdrawn from the artery, leaving the platinum coil within the dome. Depending on the size of the aneurysm, many such coils (typically from 5 to 20) may need to be placed within the dome to prevent blood from entering the aneurysm. Because pressure on the fragile dome is reduced, the risk of rupture is eliminated or greatly reduced.

Endovascular treatment permits access to vascular lesions through percutaneous introduction of microcatheters through the femoral artery, and therefore involves less patient trauma than an open surgical approach. This often results in a faster recovery and reduced morbidity and mortality.

The detachable coil shown in the drawing is shown as a coil. However, the coil can also be some other vasoocclusive form such as a braid or a combination of braids and coils. A coil is desired because it more readily severs electrolytically at a single point. Electrolytic dissolution of multi-fibered braid is complicated by the presence of multiple electrolysis points. The diameter of the wire used in such braid is typically much smaller than would be used in a coil but, again, the dissolution process is inherently more complicated. Additionally, it is within the purview of this invention to include the vasoocclusive device or connect the vasoocclusive device with fibrous materials. The fibrous materials can be materials that cause the vasoocclusive better to form a thrombus. Fibrous materials such as Dacron and the like are acceptable. Examples of fibrous adjuvants are disclosed in U.S. Pat. No. 5,226,911 to Chee et al.

The vasoocclusive coil can be pushed from the catheter into the aneurysm sac through aneurysm neck. Preferably, the detachable vasoocclusive device when in a coil form, forms a secondary loop after it leaves the end of the catheter. The most distal end of detachable coil can also have an end plug or tip of some type simply to prevent punctures of the aneurysm as it is introduced into the aneurysm sac. As noted, the detachable coil can be prebiased to form a cylinder or a conical envelope. The coil can be heat treated or crimped or otherwise physically treated to form a random shape after it is ejected from the catheter. It is desirable that a significant volume of the aneurysm be filled with the vasoocclusive device. Consequently, it is desirable that the device be quite flexible so to allow its conformance to the inner wall of the aneurysm without puncture. In any event, once the coil is properly placed within the aneurysm and the attending physician positions the electrode so to trim a proper amount of the detachable coil into the aneurysm, a modest voltage is then applied to the device. In particular, a positive electric current of approximately 0.1 to 2 milliamps at 0.1 to 5.0 volts is applied to core wire so to form a thrombus within aneurysm sac. The negative pole of power supply is attached to the conductor passing through or along the pusher.

After the thrombus has been formed and the aneurysm occluded, the core wire with its electrode is withdrawn as is the insertion end of the catheter 10. This removal typically takes place within three to ten minutes, leaving the aneurysm sac occluded.

Throughout this application, various publications, including United States patents, are referenced by author and year, and patents, by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

What is claimed is:

1. A catheter including an end portion having a platform extending substantially radially outwardly therefrom.

2. The catheter according to claim 1, wherein said platform is formed of a biocompatible expandable material for expanding in situ.

3. The catheter according to claim 2, wherein said expandable material is capable of expanding two to three times the original size.

4. The catheter according to claim 2, wherein said expandable material is a hydrophilic material.

5. The catheter according to claim 4, wherein said hydrophilic material is a hydrogel.

6. The catheter according to claim 1, wherein said platform is ring-shaped.

7. A catheter for treating aneurysms, said catheter comprising:
a lumen having an insertion end and an opposite end; and
a radially outwardly expandable ring attached to the insertion end of the catheter.

8. The catheter according to claim 7, wherein said expandable ring is formed of a biocompatible expandable material.

9. The catheter according to claim 8, wherein said expandable material is capable of expanding two to three times the original size.

10. The catheter according to claim 8, wherein said expandable material is a hydrophilic material.

11. The catheter according to claim 10, wherein said hydrophilic material is a hydrogel.

12. An expandable ring capable of being attached to a catheter.

13. The expandable ring according to claim 12, wherein said expandable ring is formed of a biocompatible expandable material.

14. The expandable ring according to claim 13, wherein said expandable material is capable of expanding two to three times the original size.

15. The expandable ring according to claim 13, wherein said expandable material is a hydrophilic material.

16. The expandable ring according to claim 15, wherein said hydrophilic material is a hydrogel.

17. A method of treating an aneurysm by inserting the catheter according to claim 1 into an artery in need of treatment.
